(19) United States
(12) Patent Application Publication
(10) Pub. No.: US 2021/0107874 A1
(43) Pub. Date: Apr. 15, 2021

(54) PHARMACEUTICAL FORMULATION OF CROTONYLAMINOPYRIDINE SALT

(71) Applicant: Intervet Inc., Madison, NJ (US)

(72) Inventors: Michael Berger, Wiesbaden (DE); Hans Peter Niedermann, Bubenheim (DE); Marcus Knell, Ockenheim (DE); Anne Lehay, Angers (FR); Angelique Duval, Angers (FR); Anne-Laure Morer, Le Plessis Grammoire (FR); Susi Alteheld, Bad Kreuznach (DE)

(73) Assignee: Intervet Inc., Madison, NJ (US)

(21) Appl. No.: 16/496,514

(22) PCT Filed: Mar. 30, 2018
(86) PCT No.: PCT/EP2018/058315

§ 371 Date: Sep. 23, 2019

(30) Foreign Application Priority Data
Mar. 31, 2017 (EP) 171642598

(51) Int. Cl. C07D 213/74; A61K 47/10; A61K 9/08; A61K 47/02; A61K 47/22; A61K 47/12; A61K 47/38; A61K 47/18; A61K 47/14; A61K 47/20; A61K 47/26

(52) U.S. Cl. CPC C07D 213/74; A61K 47/10; A61K 9/08; A61K 47/02; A61K 47/22; A61K 47/26; A61K 47/38; A61K 47/18; A61K 47/14; A61K 47/20; A61K 47/12

(57) ABSTRACT

The present invention is directed to a salt of a compound of formula (I), R¹ is halogen or methyl; X is methyl, OR², or SR²; Y¹, Y² is CR³ or N, and one of Y₁, Y₂ is N and one of Y₁, Y₂ is CR³; R² is methyl or ethyl; R³ is hydrogen or methyl, wherein the salt is chosen from the group 10 consisting of malate salt, citrate salt, tartrate salt, oxalate salt, fumarate salt, lactate salt, glucoronate salt, oxoglucoronate salt, ethanesulfonate salt, and succinate salt. The present invention is further related to a pharmaceutical formulation comprising the salt of the invention wherein the base compound is present in an amount of 2-30% (w/v) and having a pH value of at least 3.7 and to methods of treatment using the salt of the invention.

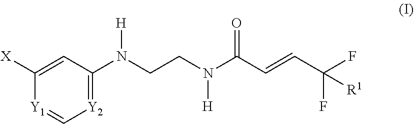

The present invention relates to a crotonylaminopyridine salt and to a pharmaceutical formulation comprising such salt.

### BACKGROUND OF THE INVENTION

Parasitic diseases in animals cause substantial suffering and economic losses throughout the world. Thus, treatment of parasitic infections remains an important global endeavor. The causative organisms include helminths, such as nematodes, cestodes, and trematodes. These organisms can infect, for example, the stomach, intestinal tract, lymphatic system, muscle tissues, kidney, liver, lungs, heart, and brain of animals.

There are many known drugs (or “anthelmintic agents”) available to treat various helminthic parasite infections. Examples of anthelminthic agents are known from WO2012/041873 and WO2013/144179. In WO2012/041873 and WO2013/144179 formulations of some compounds with organic solvents are disclosed. Also in general it is indicated that the compounds may in a salt form, however no specific salt forms or aqueous formulations are discloses in WO2012/041873 and WO2013/144179.

There exists a need for novel forms of anthelmintic agents that may be easily formulated such as in aqueous formulation and for formulations that are suitable for injection.

### SUMMARY OF THE INVENTION

The present invention is directed to a salt of a compound of formula (I)

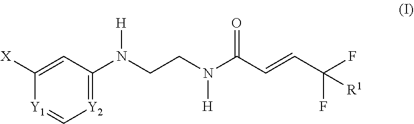

R¹ is halogen or methyl; X is methyl, OR², or SR²; Y₁, Y₂ is CR³ or N, and one of Y₁, Y₂ is N and one of Y₁; Y₂ is CR³; R² is methyl or ethyl; R³ is hydrogen or methyl, and wherein the salt is chosen from the group consisting of malate salt, citrate salt, tartrate salt, oxalate salt, fumarate salt, lactate salt, glucoronate salt, oxoglucoronate salt, ethanesulfonate salt, and succinate salt.

Suitably, the compounds of formula (I) may be selected from the following compounds

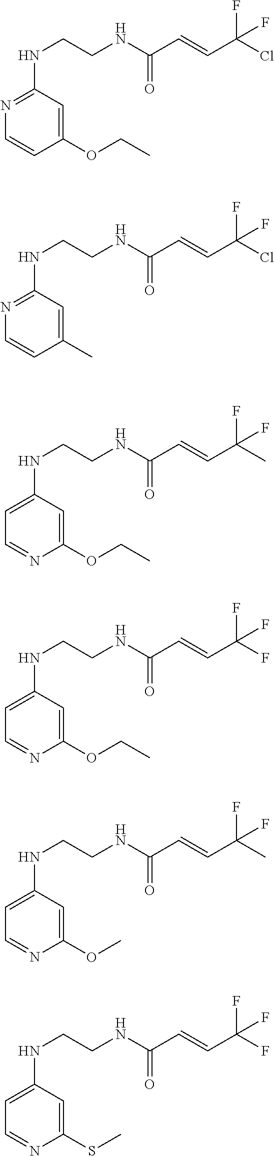 [SMILES: CCOc1ccnc(NCCNC(=O)/C=C/C(F)(F)Cl)c1 ; Cc1ccnc(NCCNC(=O)/C=C/C(F)(F)Cl)c1 ; CCOc1cc(NCCNC(=O)/C=C/C(C)(F)F)ccn1 ; CCOc1cc(NCCNC(=O)/C=C/C(F)(F)F)ccn1 ; COc1cc(NCCNC(=O)/C=C/C(C)(F)F)ccn1 ; CSc1cc(NCCNC(=O)/C=C/C(F)(F)F)ccn1]

A suitable salt according to the invention is a salt chosen from the group consisting of malate salt, citrate salt, tartrate salt, oxalate salt, fumarate salt, oxoglucoronate salt, and ethanesulfonate salt. More suitably the salt is chosen from the group consisting of malate salt, citrate salt, and tartrate salt.

The invention is further related to a pharmaceutical formulation comprising the salt of the invention and/or embodiments thereof. Suitably the salt of the invention and/or embodiments thereof is present in the pharmaceutical formulation in an amount of 2-30% (w/v) based on the weight of the base compound. Suitably the formulation has a pH value of at least 3.7.

Suitably the formulation is an aqueous formulation. Suitable pharmaceutical formulations according to the invention and/or embodiments thereof comprise a preservative. Suitably the preservative is benzylalcohol.

Suitably the pharmaceutical formulation according to the invention and/or embodiments thereof further comprises an antifreeze compound. Suitably the antifreeze compound is selected from the group consisting of propylene glycol, ethylene glycol, and glycerol, suitably the antifreeze compound is propylene glycol.

In a preferable embodiment, the pharmaceutical composition according to the invention and/or embodiments thereof is suitable for injection.

The salt and the formulation of the invention and/or embodiments thereof may also be used in a method of treatment. The salt and the formulation of the invention and/or embodiments are very suitable for treatment of parasitic diseases in animals such as a helminth infection in an animal.

### DETAILED DESCRIPTION

The present invention is directed to a salt of a compound of formula (I)

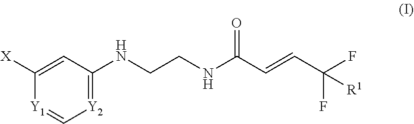

These compounds may be used to treat parasitic diseases in animals. Compounds of formula (I) are known from WO2012/041873 and from WO2013/144179. WO2012/041873 and WO2013/144179 disclose methods how to synthesise the compounds.

The present invention is directed to a selection of the compounds of WO2012/041873 and WO2013/144179 where R¹ is halogen or methyl; X is methyl, OR², or SR²; Y₁, Y₂ is CR³ or N, and one of Y₁, Y₂ is N and one of Y₁; Y₂ is CR³; R² is methyl or ethyl; R³ is hydrogen or methyl.

Surprisingly it was found that when these compounds were formulated as a specific salt, the compounds could be formulated displaying favorable pharmakinetic properties. It was found that the malate salt, citrate salt, tartrate salt, oxalate salt, fumarate salt, lactate salt, glucoronate salt, oxoglucoronate salt, ethanesulfonate salt, or succinate salt of the compounds as described above were easy to dissolve and displayed favorable pharmakinetic properties.

Suitably the halogen may be fluorine or chlorine, preferably fluorine. Suitably, the SR² is S-methyl. Suitably OR² is O-methyl or O-ethyl. Suitably R³ is hydrogen. Suitably Y₁ is N and Y₂ is CH.

Suitably, the compounds of formula (I) may be selected from the following compounds

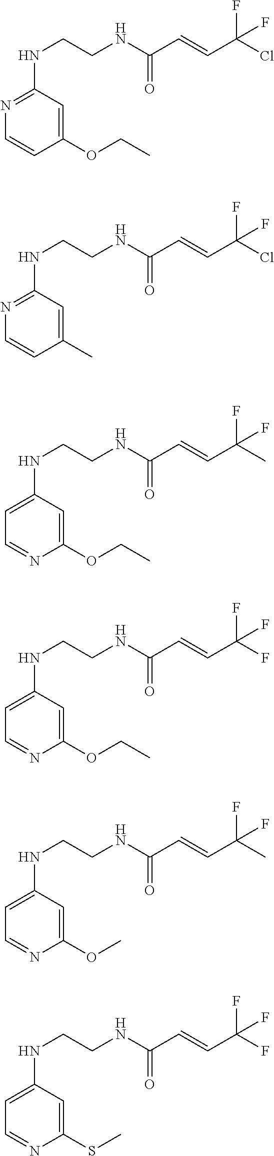 [SMILES: CCOc1ccnc(NCCNC(=O)/C=C/C(F)(F)Cl)c1 ; Cc1ccnc(NCCNC(=O)/C=C/C(F)(F)Cl)c1 ; CCOc1cc(NCCNC(=O)/C=C/C(C)(F)F)ccn1 ; CCOc1cc(NCCNC(=O)/C=C/C(F)(F)F)ccn1 ; COc1cc(NCCNC(=O)/C=C/C(C)(F)F)ccn1 ; CSc1cc(NCCNC(=O)/C=C/C(F)(F)F)ccn1]

Suitable salt according to the invention is a salt chosen from the group consisting of malate salt, citrate salt, tartrate salt, oxalate salt, fumarate salt, oxoglucoronate salt, and ethanesulfonate salt. More suitably the salt is chosen from the group consisting of malate salt, citrate salt, and tartrate salt.

Good pharmacokinetic properties were obtained with the tartrate salt of

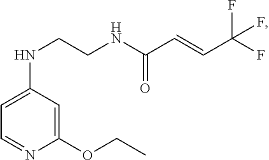 [SMILES: CCOc1cc(NCCNC(=O)/C=C/C(F)(F)F)ccn1]

with the tartrate salt of

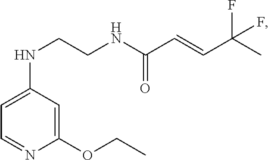 [SMILES: CCOc1cc(NCCNC(=O)/C=C/C(C)(F)F)ccn1]

with the tartrate salt of

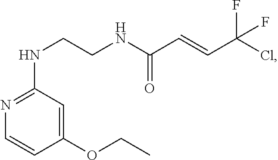 [SMILES: CCOc1ccnc(NCCNC(=O)/C=C/C(F)(F)Cl)c1]

with the tartrate salt of

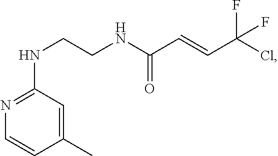 [SMILES: Cc1ccnc(NCCNC(=O)/C=C/C(F)(F)Cl)c1]

with the tartrate salt of

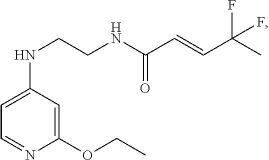 [SMILES: CCOc1cc(NCCNC(=O)/C=C/C(C)(F)F)ccn1]

with the tartrate salt of

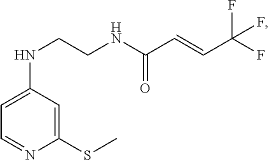 [SMILES: CSc1cc(NCCNC(=O)/C=C/C(F)(F)F)ccn1]

with the citrate salt of

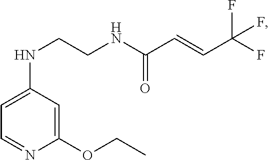 [SMILES: CCOc1cc(NCCNC(=O)/C=C/C(F)(F)F)ccn1]

with the citrate salt of

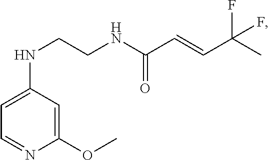 [SMILES: COc1cc(NCCNC(=O)/C=C/C(C)(F)F)ccn1]

with the citrate salt of

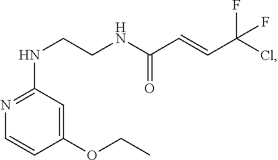 [SMILES: CCOc1ccnc(NCCNC(=O)/C=C/C(F)(F)Cl)c1]

with the citrate salt of

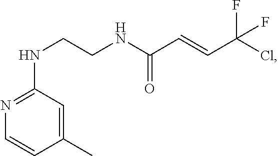 [SMILES: Cc1ccnc(NCCNC(=O)/C=C/C(F)(F)Cl)c1]

with the citrate salt of

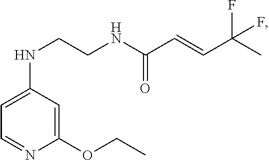 [SMILES: CCOc1cc(NCCNC(=O)/C=C/C(C)(F)F)ccn1]

with the citrate salt of

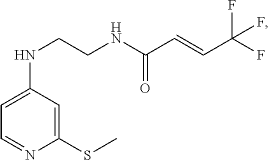 [SMILES: CSc1cc(NCCNC(=O)/C=C/C(F)(F)F)ccn1]

with the malate salt of

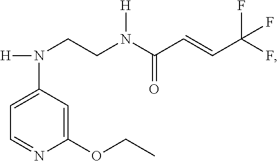 [SMILES: CCOc1cc(NCCNC(=O)/C=C/C(F)(F)F)ccn1]

with the malate salt of

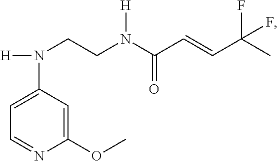 [SMILES: COc1cc(NCCNC(=O)/C=C/C(C)(F)F)ccn1]

with the malate salt of

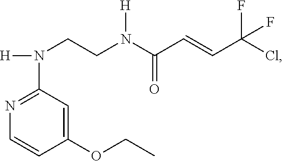 [SMILES: CCOc1ccnc(NCCNC(=O)/C=C/C(F)(F)Cl)c1]

with the malate salt of

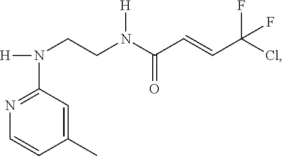 [SMILES: Cc1ccnc(NCCNC(=O)/C=C/C(F)(F)Cl)c1]

with the malate salt of and

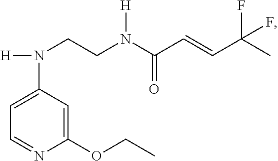 [SMILES: CCOc1cc(NCCNC(=O)/C=C/C(C)(F)F)ccn1]

with the malate salt of

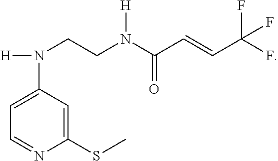 [SMILES: CSc1cc(NCCNC(=O)/C=C/C(F)(F)F)ccn1]

Good pharmacokinetic properties were obtained with the tartrate salt of

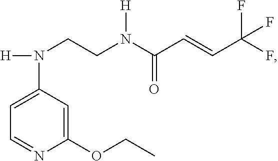 [SMILES: CCOc1cc(NCCNC(=O)/C=C/C(F)(F)F)ccn1]

with the tartrate salt of

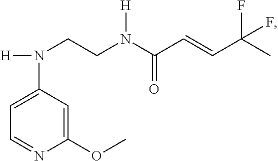 [SMILES: COc1cc(NCCNC(=O)/C=C/C(C)(F)F)ccn1]

with the citrate salt of

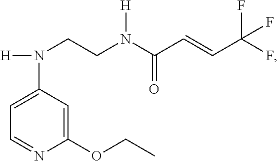 [SMILES: CCOc1cc(NCCNC(=O)/C=C/C(F)(F)F)ccn1]

with the citrate salt of

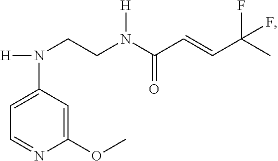 [SMILES: COc1cc(NCCNC(=O)/C=C/C(C)(F)F)ccn1]

with the malate salt of

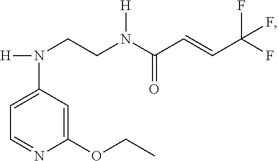 [SMILES: CCOc1cc(NCCNC(=O)/C=C/C(F)(F)F)ccn1]

and with the malate salt of

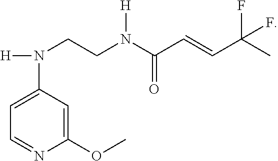 [SMILES: COc1cc(NCCNC(=O)/C=C/C(C)(F)F)ccn1]

The salt of the invention may be synthesized as salt. A skilled person is well aware of the processes to synthesize salts. The salt of the invention may also be formed in situ in a solution of the base compound as described in formula (I) and an acid to form the salt. In general, an acid addition salt can be prepared by reacting a free base compound with an approximately stoichiometric amount of the acid. Suitably the acid and base compound are added to an aqueous solution in a molar ratio of 2:1 to 1:2. Suitably the base compound and the acid are added in a molar ratio of 1:1. Suitably the amount of acid is 10 to 300 mg/ml, more suitably 20 to 250 mg/ml, more suitable 30 to 220 mg/ml, more suitably 35 to 200 mg/ml, more suitably 40 to 180 mg/ml, more suitably between 45 and 150 mg/ml, more suitably between 50 and 125 mg/ml, more suitably between 55 and 120 mg/ml, more suitably between 60 and 110 mg/ml, more suitably between 65 and 100 mg/ml, more suitably between 70 and 95 mg/ml, more suitably between 75 and 90 mg/ml. Depending on the acid and the pH for the solution, a skilled can easily vary the amount of acid for each compound to obtain the desired salt and solution thereof.

The invention is further related to a pharmaceutical formulation comprising the salt of the invention and/or embodiments thereof. Suitably the salt of the invention and/or embodiments thereof is present in the pharmaceutical formulation such that the base compound is present in the formulation in an amount of 2-30% (w/v). As the counter ion of the salt may vary the amount of salt in the formulation is most suitably determined on the amount of base compound. Suitably the salt of the invention and/or embodiments thereof is present in the pharmaceutical formulation such that the base compound is present in the formulation in an amount of 3-25% (w/v), more suitably in an amount of 4-22% (w/v), more suitably in an amount of 5-20% (w/v), more suitably in an amount of 6-18% (w/v), more suitably in an amount of 7-15% (w/v), more suitably in an amount of 8-12% (w/v), even more suitably in an amount of 9-10% (w/v).

Suitably the formulation has a pH value of at least 3.7. More suitably the formulation has a pH of at least 4.0, more suitably a pH of at least 4.2 more suitably a pH of at least 4.3, more suitably a pH of at least 4.4. Suitably, the formulation has a pH value of between 3.7 and 7.4, more suitably of between 4.0 and 7.0, more suitably of between 4.3 and 6.5, more suitably of between 4.4 and 6.0, and more suitably between 4.5 and 5.5. Suitably the formulation has a pH value of between 4.0 and 4.4.

The pH of the formulation may be adjusted with pH control agents such as sodium hydroxide, ammonium hydroxide, potassium hydroxide, ammonia, barium hydroxide, calcium hydroxide, sodium carbonate, urea, guanidium, oxides such as copper oxide, magnesium oxide, hexamine, sodium amide, lactic acid, citric acid, or acetic acid. Suitably pH control agents are ammonium hydroxide, sodium carbonate, lactic acid, citric acid, or acetic acid.

Suitably the formulation is an aqueous formulation.

Suitable pharmaceutical formulation according to the invention and/or embodiments thereof comprises a preservative. The preservative may be selected from the group consisting of ascorbic acid, citric acid, sodium benzoate, calcium propionate, sodium nitrite, calcium sorbate, potassium sorbate, benzyl alcohol, butylated hydroxyanisole (BHA), Butylated hydroxytoluene (BHT), Ethylenediaminetetraacetic acid (EDTA), and tocopherols (Vitamin E). Suitably the preservative is benzylalcohol.

Suitably the preservative is present in an amount of 0.1-20% (w/v), more suitably in an amount of 0.2-15% (w/v), more suitably in an amount of 0.5-12% (w/v), more suitably in an amount of 0.7-10% (w/v), more suitably from 0.8-8% (w/v), more suitably from 1-7% (w/v), more suitably from 1.2-5% (w/v), more suitably from 1.5-4% (w/v) more suitably from 1.7-3% (w/v), and more suitably from 2-2.5% (w/v). Suitably ranges are from 0.2 to 5%, from 0.3 to 4.5%, from 0.4 to 4%, from 0.5, 3.5%, from 0.6 to 3%, from 0.7 to 2.7%, from 0.8 to 2.5%, from 0.9 to 2.3% and from 1 to 2%.

Suitably benzylalcohol is present in an amount of 0.1-20% (w/v), more suitably in an amount of 0.2-15% (w/v), more suitably in an amount of 0.5-12% (w/v), more suitably in an amount of 0.7-10% (w/v), more suitably from 0.8-8% (w/v), more suitably from 1-7% (w/v), more suitably from 1.2-5% (w/v), more suitably from 1.5-4% (w/v) more suitably from 1.7-3% (w/v), and more suitably from 2-2.5% (w/v). Suitably ranges are from 0.2 to 5%, from 0.3 to 4.5%, from 0.4 to 4%, from 0.5, 3.5%, from 0.6 to 3%, from 0.7 to 2.7%, from 0.8 to 2.5%, from 0.9 to 2.3% and from 1 to 2%.

Suitably the pharmaceutical formulation according to the invention and/or embodiments thereof further comprises an antifreeze compound. Suitably the antifreeze compound is selected from the group consisting of propylene glycol, ethylene glycol, and glycerol. Suitably the antifreeze compound is propylene glycol. Suitably the antifreeze component is present in an amount of 10-80% (w/v), more suitably in an amount of 20-70% (w/v), more suitably in an amount of 25-60% (w/v), more suitably in an amount of 30-50% (w/v), or more suitably in an amount of 35-40% (w/v). A suitably amount of antifreeze compound is from 30-40% (w/v).

Suitably propylene glycol is present in an amount of 10-80% (w/v), more suitably in an amount of 20-70% (w/v), more suitably in an amount of 25-60% (w/v), more suitably in an amount of 30-50% (w/v), or more suitably in an amount of 35-40% (w/v). A suitably amount of propylene glycol is from 30-40% (w/v).

In a preferable embodiment, the pharmaceutical composition according to the invention and/or embodiments thereof is suitable for injection.

The formulation of the present invention may further comprise pharmaceutically acceptable excipients. Such excipients are added to the formulations for a variety of purposes. One or more pharmaceutically acceptable excipients may be present in the composition of the present invention, such as for example diluents, binders, lubricants, disintegrants, glidants, and addifying agents.

The term “excipient” shall mean a pharmacologically inactive substance which is used as a carrier for the active substance and/or the design of formulations of drug products. The term “excipient” shall include a pharmaceutically acceptable, pharmacologically inactive ingredient such as a binder, a filler, a coating-forming compound, a plasticizers for coatings and a compound which masks odors. Some examples of optional excipients are pigments, disintegrants, antioxidants, flavors, sweeteners, colourants, opacifiers, anti-adhesives, preservatives, glidants, lubricants, sorbents and isolating-layer forming agents. Suitable substances are known in the art. The term “excipient” applied to pharmaceutical formulations of the invention also refers to a diluent or vehicle with which an active substance is administered. Such pharmaceutical excipients can be from animal, vegetable or synthetic origin.

The term “pharmaceutically acceptable” in connection with a substance shall mean an ingredient or a substance which does not affect the safety of a human and/or animal and/or is well-tolerated by a human and/or animal after administration.

Suitable pharmaceutically acceptable excipients of the formulations of the present invention include: povidone, sodium hydroxide, isopropyl alcohol, silicified MCC90 (Frosolv®), colloidal silicon dioxide (Aerosil®), dibasic calcium phosphate hydrous, croscarmellose sodium, magnesium oxide heavy, magnesium stearate, microcrystalline cellulose and iron oxides.

Suitable diluents include for example pharmaceutically acceptable inert fillers such as microcrystalline cellulose, lactose, pregelatinized starch, dibasic calcium phosphate, saccharides, and/or mixtures of the foregoing.

The pharmaceutical formulation may comprise preservatives and/or antioxidants. Useful examples of preservatives and antioxidants are sorbic acid, sodium sorbate and potassium sorbate, methyl-p-hydroxybenzoate (methylparaben), ethyl-p-hydroxybenzoate (ethylparaben), and propyl-p-hydroxybenzoate (propylparaben), ascorbic acid, benzyl alcohol, sodium ascorbate or potassium ascorbate, gallic acid and sodium or potassium gallates, or mixtures thereof. Contemplated preservatives include, for example, phenol, alkyl esters of parahydroxybenzoic acid (e.g., methyl p-hydroxybenzoate (or “methylparaben”) and propyl p-hydroxybenzoate (or “propylparaben”)), sorbic acid, o-phenylphenol benzoic acid and the salts thereof, chlorobutanol, benzyl alcohol, thimerosal, phenylmercuric acetate and nitrate, nitromersol, benzalkonium chloride, and cetylpyridinium chloride. Benzylalcohol is particularly preferred.

The pharmaceutical formulation may comprise buffering agents. Suitable examples of buffering agents are organic and inorganic acid-base buffer systems, for example, citric acid and citrates of sodium or potassium, phosphoric acid and phosphates of sodium and of potassium. The combination citric acid and sodium citrate is preferred as well as the combination of acetic acid and sodium acetate. Contemplated buffers include, for example, sodium citrate, and magnesium and calcium carbonate and bicarbonate.

The pharmaceutical formulation may comprise stabilizers. Useful examples of stabilizers include alginic acid and alginates of sodium and potassium, agar-agar, carrageenin, and gum tragacanth, carboxymethylcellulose, sorbitol, pectin, xanthan gum, isomalt, lecithin, phospholipid, malitol, maltodextrin. Contemplated stabilizers include, for example, chelating agents and antioxidants.

The pharmaceutical formulation may comprise polyethylene glycol. Suitable examples of polyethylene glycol is polyethylene glycol of high molecular weight, preferably greater than 1,000, more preferably between 2,000 and 10,000, and even more preferably between 3,000 and 8,000. Typically, the polyethylene glycol that may be used in the pharmaceutical formulation of the present invention is PEG 4000 or PEG 6000. The polyethylene glycols that can be used in the present invention are commercial products, distributed for example by Alfa Aesar GmbH, Karlsruhe, Germany, and CarboMer, Inc., San Diego, Calif., USA. Preferably, the pharmaceutical formulation of the present invention includes an amount of polyethylene glycol greater than 10% w/v, and more preferably greater than 15% w/v relative to the total volume of said pharmaceutical formulation. Typically, the optimum amount of polyethylene glycol included in the liquid pharmaceutical formulation of the present invention may be between 15% and 20% w/v relative to the total volume of said pharmaceutical formulation.

Advantageously, the formulation may comprise polyalcohols. Suitable examples are polyalcohols selected from polyols and/or polyalkyleneglycols having solubilising, surfactant and sweetening properties as main functions.

Suitably, said polyols are selected from glycols, such as ethylene glycol, propylene glycol, 1,3-propanediol, trimethylene glycol, 1,2-butanediol, 1,3-butanediol, 1,4-butanediol, 1,2-pentanediol, 1,4-pentanediol, 1,5-pentanediol, 1,6-hexanediol, 1,7-heptanediol, diethylene glycol, dipropylene glycol, glycerol, 1, 1, 1-trimethylolpropane, 1,1,1-trimethylolethane, 1,2,6 hexanetriol, etohexadiol, 2-methyl-2,4-pentanediol, 1,8-octanediol, and glycerol; sugar alcohols, such as threitol, arabitol, ribitol, dulcitol, iditol, lactitol, pentaerythritol, maltitol, sorbitol, mannitol, xylitol, erythritol, isomalt; and sugars, such as ribose, arabinose, xylose, lyxose, deoxyribose, fructose, sorbose, glucose, mannose, galactose, gulose, sucrose, lactose, lactulose, maltose, trehalose, turanose, cellobiose, and mixture thereof.

More suitably, the polyols useful in the present invention are selected from glycerol, propylene glycol, xylitol, sorbitol, sucrose, glucose, and mixture thereof.

Suitably, said polyalkylene glycols are selected from polyethylene glycol, polypropylene glycol, esters thereof with organic acids, or ethers thereof with alcohols, and mixture thereof.

More preferably, the polyalkylene glycols useful in the present invention are selected from esters of polyethylene glycols having a molecular weight ranging from 200 to 1500 (PEG 200, PEG 300, PEG 400, PEG 600, PEG 660, PEG 1000, PEG 1500) with hydroxy fatty acids, such as, for example hydroxylauric acid, hydroxymyristic acid, hydroxypalmitic acid, hydroxystearic acid, and hydroxyarachidic acid.

Advantageously, the polyalkylene glycol ester useful in the present invention is Solutol™ HS 15 (Macrogol 15 hydroxystearate, Ph. Eur., Monograph 2052), a polyethylene glycol 660 hydroxystearate manufactured by BASF (Parsippany, N.J.).

The total content of the above described polyols and polyalkylene glycols may be at least 20% by weight based on the total volume of the aqueous solution (w/v). Preferably, the total content of the above described polyols and polyalkylene glycols ranges from 25% to 75% w/v, more preferably from 30% to 70% w/v, and most preferably from 35% to 65% w/v. Particularly, the total content of alcohols is lower than 0.7% (w/v), and more particularly, the presence of alcohols may also be totally avoided.

The pharmaceutical formulation may comprise surfactants. Suitable examples of surfactants are in particular ethoxylated esters of sorbitan fatty acids, block polymers and block copolymers (such as poloxamers and poloxamines), polyglycerol ethers and polyglycerol esters, lecithins of various origins (such as egg lecithin or soya lecithin), chemically modified lecithins (such as hydrogenated lecithins), as well as phospholipids and sphingolipids, mixtures of lecithins with phospholipids, sterols (such as cholesterol and its derivatives, specifically stigmasterol), esters and ethers of sugars or of sugar alcohols with fatty acids or fatty alcohols (such as saccharose monostearate), polysorbate, e.g., which is an emulsifier derived from PEG-ylatedsorbitan (a derivative of sorbitol) esterified with fatty acids. This class of agents comprises, among others, polysorbates 20, 21, 40, 60, 61, 65, 80, 81, 85, and 120. More preferably, polysorbate 80 (polyoxyethylene(20)-sorbitan-monooleate) or polysorbate 20 (polyoxyethylene(20)-sorbitan-monolaurate) may be used. Contemplated surfactants include, for example, polyoxyethylene sorbitan fatty acid esters; polyoxyethylene monoalkyl ethers; sucrose monoesters; lanolin esters and ethers; alkyl sulfate salts; and sodium, potassium, and ammonium salts of fatty acids.

The pharmaceutical formulation may comprise sterically stabilizing substances. Suitable examples of sterically stabilizing substances are poloxamers and poloxamines (block copolymers of polyoxyethylene and polyoxypropylene), ethoxylated esters of sorbitan fatty acids, in particular polysorbates (such as polysorbate 80 or Tween 80®), ethoxylated mono- and diglycerides, ethoxylated lipids, ethoxylated fatty alcohols and fatty acids.

The pharmaceutical formulation may comprise charged ionic stabilizers and peptizing agents. Suitable examples of charged ionic stabilizers and peptizing agents are diacetyl phosphates, phosphatidyl glycerol, as well as saturated and unsaturated fatty acids, sodium cholate, sodium glycocholate, sodium taurocholate or mixtures thereof, aminoacids or peptising agents such as sodium citrate.

Charge stabilizers if necessary or desired, are preferably added in the amount of 0.01% to 10% (w/v), and in particular between 0.05% and 2%, of the basic formulation.

The pharmaceutical formulation may comprise viscosity-increasing substances. Suitable examples of viscosity-increasing substances are cellulose ethers and cellulose esters (e.g. methyl cellulose, hydroxyethylcellulose, hydroxypropylcellulose, sodium carboxymethylcellulose), polyvinyl derivatives such are polyvinyl alcohol, polyvinyl pyrrolidone, polyvinyl acetate, alginates, polyacrylates (such as Carbopol®), xanthanes and pectins. Contemplated viscosity-enhancing agents include, for example, polyethylene, methylcellulose, sodium carboxymethylcellulose, hydroxypropyl-methylcellulose, hydroxypropylcellulose, sodium alginate, carbomer, povidone, acacia, guar gum, xanthan gum, tragacanth, methylcellulose, carbomer, xanthan gum, guar gum, povidone, sodium carboxymethylcellulose, magnesium aluminum silicate, carboxyvinyl polymers, carrageenan, hydroxyethyl cellulose, laponite, water-soluble salts of cellulose ethers, natural gums, colloidal magnesium aluminum silicate or finely divided silica, homopolymers of acrylic acid crosslinked with an alkyl ether of pentaerythritol or an alkyl ether of sucrose, and carbomers.

Viscosity-increasing agents, if necessary or desired, are incorporated in a comparable ratio preferably in the amount of 0.01-10%, especially in the amount of between 0.1% and 10% (w/v), and preferably between 0.5% and 5% of the formulation.

The pharmaceutical formulation may comprise binders. Suitable binders include, for example, the following: povidone; copovidone; alginic acid; sodium alginate; cellulose derivatives such as hydroxypropyl memylcellulose, hydroxy propyl cellulose, hydroxyethylcellulose, methylcellulose and ethyl cellulose; gelatin; starch or starch derivatives; and mixtures thereof. Contemplated binders include, for example, gelatin, acacia, and carboxymethyl cellulose.

The pharmaceutical formulation may comprise lubricants. Suitable lubricants include, for example, the following: magnesium-, or calcium-stearate, stearic acid, sodium stearyl fumarate, talc, sodium benzoate, glyceryl mono fatty acid, glyceryl monostearate hydrogenated vegetable oil, polyethylene glycol, and mixtures thereof. Contemplated lubricants include, for example, magnesium stearate, stearic acid, and talc.

The pharmaceutical formulation may comprise disintigrants. Suitable disintegrants include, for example, the following: croscarmellose sodium, sodium starch glycolate, maize starch, carboxymethylcellulose, microcristalline cellulose, cross-linked polyvinylpyrrolidone, alginic acid, sodium alginate, pregelatinized starch and low-substituted hydroxypropylcellulose, Contemplated disintegrants include, for example, corn starch, alginic acid, sodium carboxymethylcellulose, and sodium croscarmellose.

The pharmaceutical formulation may comprise anti-adherents. Suitable anti-adherents include, for example, one or more compounds that are capable of preventing stickiness to surfaces of the vial. Examples of anti-adherents include silicon containing compounds such as colloidal silicon dioxide, magnesium trisilicate and talc.

Other inert ingredients may generally be added to the composition as desired. To illustrate, it is contemplated that these may include, for example, lactose, mannitol, sorbitol, calcium carbonate, sodium carbonate, tribasic calcium phosphate, dibasic calcium phosphate, sodium phosphate, kaolin, compressible sugar, starch, calcium sulfate, dextro or microcrystalline cellulose, colloidal silicon dioxide, starch, sodium starch glycolate, crospovidone, microcrystalline cellulose, tragacanth, hydroxypropylcellulose, pregelatinized starch, povidone, ethylcellulose, hydroxypropylcellulose, hydroxypropylmethylcellulose, and methylcellulose.

Further aspects regarding formulation of drugs and various excipients are found in, for example, Gennaro, A. R., et al., eds., Remington: The Science and Practice of Pharmacy (Lippincott Williams & Wilkins, 20th Ed., 2000). Another source regarding formulation of drugs and various excipients is found in, for example, Liberman, H. A., et al., eds., Pharmaceutical Dosage Forms (Marcel Decker, New York, N.Y., 1980).

The concentration of the salts according to this invention in the applied dosage form may vary widely depending on, for example, the dosage route. In general, the concentration is from about 1 to about 70% (by weight). In some such embodiments, for example, the concentration is from about 1 to about 50% (by weight), or from about 10 to about 50% (by weight). In other embodiments, the concentration is from about 35 to about 65% (by weight), from about 40 to about 60% (by weight), from about 45 to about 55% (by weight), or about 50% (by weight).

Suitable routes of administration may, for example, include oral, rectal, transdermal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Suitably the administration is parenteral. Suitably the administration is by injection. Suitably the administration is subcutaneous or intravenous.

Injectable formulations may be prepared according to, for example, the known art using suitable solvents, solubilizing agents, protecting agents, dispersing agents, wetting agents, and/or suspending agents. Contemplated carrier materials include, for example, water, ethanol, butanol, benzyl alcohol, glycerin, 1,3-butanediol, Ringer's solution, isotonic sodium chloride solution, bland fixed oils (e.g., synthetic mono- or diglycerides), vegetable oil (e.g., corn oil), dextrose, mannitol, fatty acids (e.g., oleic acid), dimethyl acetamide, surfactants (e.g., ionic and non-ionic detergents), N methylpyrrolidone, propylene glycol, and/or polyethylene glycols (e.g., PEG 400). Contemplated solubilizing agents include, for example, polyvinyl pyrrolidone, polyoxyethylated castor oil, polyoxyethylated sorbitan ester, and the like. Contemplated protecting agents include, for example, benzyl alcohol, trichlorobutanol, phydroxybenzoic acid ester, n-butanol, and the like.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

The compounds and pharmaceutical compositions according to this invention are useful in treating parasitic infections such as helminth infections of animals. An “effective amount,” is the amount or quantity of a compound that is required to alleviate or reduce parasite numbers in an animal, and/or to inhibit the development of parasite infections in an animal, in whole or in part.

This amount is readily determined by observation or detection of the pathogen numbers such as parasite numbers both before and after contacting the sample of pathogens such as parasites including their stages with the salt according to this invention, directly and/or indirectly, e.g., by contacting articles, surfaces, foliage, or animals with the compound.

This can be evaluated by counting parasites (especially helminthes) directly after necroscopy of the host animal.

The reduction of parasite numbers, especially gastrointestinal helminth parasites can be alternatively measured in-directly by faecal egg or differential larval counts. In this case the effective amount of the compound is determined by the reduction of the number of excreted helminth eggs or larvae in the faeces of the treated animal before and after treatment. For an in vivo administration the salt according to this invention, is preferably administered to an animal in an effective amount which is synonymous with “pharmaceutically effective amount” or “anthelmintically effective amount”.

A single administration of a salt according to this invention is typically sufficient to treat a parasitic infection such as a helminth infection, preferably a nematode, cestode or trematode infection, more preferably a nematode infection. Although such a single dose is typically preferred, it is contemplated that multiple doses can be used. When the salt according to this invention is orally administered, the total dose to treat a disease such as a helminth infection is generally greater than about 0.01 mg/kg (i.e., milligram of salt according to this invention per kilogram body weight of the treated animal). In some such embodiments, the total dose is from about 0.01 to about 100 mg/kg, from about 0.01 to about 50 mg/kg, from about 0.1 to about 25 mg/kg, or from about 1 to about 20. For sheep, for example, the dose is generally from about 0.5 to about 15 mg/kg, from about 1 to about 10 mg/kg. The same dose range may be suitable for other dosage routes. For example, in some embodiments, the same dose range is used for subcutaneous administration. The desired dose, however, may be less in some instances where the salt according to this invention is administered intravenously.

If the salt according to this invention is administered parenterally via an injection, the concentration of the salt according to this invention in the dosage form preferably is sufficient to provide the desired therapeutically effective amount of the salt according to this invention in a volume that is acceptable for parenteral administration.

Factors affecting the preferred dosage may include, for example, the parasite species infection to be treated and the development stages of the parasites, the type (e.g., species and breed), age, size, sex, diet, activity, and condition of the of the infected animal; the dosage route; pharmacological considerations, such as the activity, efficacy, pharmacokinetic, and toxicology profiles of the particular composition administered; and whether the salt according to this invention being administered as part of a combination of active ingredients. Thus, the preferred amount of the salt according to this invention can vary, and, therefore, can deviate from the typical dosages set forth above. Determining such dosage adjustments is generally within the skill of those in the art.

The salts according to the invention and embodiments thereof are for use as a medicament for animals. In some embodiments of this invention, one or more, preferably one salt according to this invention is administered to treat infections, in particular a helminth infection of an animal. In one embodiment one or more, preferably one salt according to this invention is administered to treat parasitoses of an animal.

The term “(parasitic) infection” includes conditions associated with or caused by one or more (parasitic) pathogens; said conditions include clinical conditions (parasitoses) and sub-clinical conditions. The term “treatment of parasitic infection” thus includes both the treatment of parasitoses and the treatment of sub-clinical conditions. The treatment of a parasite infection generally implies the suppression of parasite (e.g. helminth) burdens in the animal below that level at which economic loss occurs.

Sub-clinical conditions are typically conditions not directly leading to clinical symptoms in the parasite infected animal but leading to economic losses. Such economic losses can be e.g. by depression of growth in young animals, lower feed efficiency, lower weight gain in meat producing animals, lower milk production in ruminants, lower egg production in laying hens, or lower wool-production in sheep.

The term “parasitoses” relates to clinically manifest pathologic conditions and diseases associated with or caused by an infection by one or more parasites, such as, for example parasitic gastroenteritis or anemia in ruminants e.g. sheep and goats or colic in horses.

In general, the prevention or treatment of parasitic infection including parasitoses is achieved by administering one or more, preferably one salt according to this invention to treat a parasitic infection such as a helminth infection.

Thus the invention provides a method of treating a (parasitic) infection such as a helminth infection, including parasitoses, which comprises administering to the animal an antiparasitically, preferably an anthelmintically, effective amount of one or more salts according to this invention. Preferably nematode, cestode or trematode infections are treated, more preferably nematode infections.

“Treating (parasitic) infections” includes treating parasitoses and means to partially or completely inhibit the development of (parasitic) infections of an animal susceptible to (parasitic) infection, reduce or completely eliminate the symptoms of infections of an animal having infections, and/or partially or completely cure infections of an animal having infections. This can be achieved by alleviating or reducing pathogen numbers such as parasite numbers in an animal.

The effect of the salts according to this invention can be e.g. ovicidal, larvicidal, and/or adulticidal or a combination thereof. The effect can manifest itself directly, i.e. killing the parasites either immediately or after some time has elapsed, for example when malting occurs, or by destroying their eggs, or indirectly, e.g. reducing the number of eggs laid and/or the hatching rate. Alternatively the parasite is not killed but paralyzed and is then dislodged and excreted by the host animal.

The salts and pharmaceutical compositions according to this invention are useful in treating parasitic infections such as helminth infections of animals. An “effective amount,” is the amount or quantity of a salt that is required to alleviate or reduce parasite numbers in an animal, and/or to inhibit the development of parasite infections in an animal, in whole or in part.

This amount is readily determined by observation or detection of the pathogen numbers such as parasite numbers both before and after contacting the sample of pathogens such as parasites including their stages with the salt according to this invention, directly and/or indirectly, e.g., by contacting articles, surfaces, foliage, or animals with the salt e.g. the parasite count is reduced, after a first administration, by an amount ranging from 5% to about 100%.

This can be evaluated by counting parasites (especially helminthes) directly after necroscopy of the host animal.

The reduction of parasite numbers, especially gastrointestinal helminth parasites can be alternatively measured in-directly by faecal egg or differential larval counts. In this case the effective amount of the salt is determined by the reduction of the number of excreted helminth eggs or larvae in the faeces of the treated animal before and after treatment. For an in viva administration the salt according to this invention, is preferably administered to an animal in an effective amount which is synonymous with “pharmaceutically effective amount” or “anthelmintically effective amount”.

A single administration of a salt according to this invention is typically sufficient to treat a parasitic infection such as a helminth infection, preferably a nematode, cestode or trematode infection, more preferably a nematode infection. Although such a single dose is typically preferred, it is contemplated that multiple doses can be used. When the salt according to this invention is orally administered, the total dose to treat a disease such as a helminth infection is generally greater than about 0.01 mg/kg (i.e., milligram of salt according to this invention per kilogram body weight of the treated animal). In some such embodiments, the total dose is from about 0.01 to about 100 mg/kg, from about 0.01 to about 50 mg/kg, from about 0.1 to about 25 mg/kg, or from about 1 to about 20. For sheep, for example, the dose is generally from about 0.5 to about 15 mg/kg, from about 1 to about 10 mg/kg. The same dose range may be suitable for other dosage routes. For example, in some embodiments, the same dose range is used for subcutaneous administration. The desired dose, however, may be less in some instances where the salt according to this invention is administered intravenously.

If the salt according to this invention is administered parenterally via an injection, the concentration of the salt according to this invention in the dosage form preferably is sufficient to provide the desired therapeutically effective amount of the salt according to this invention in a volume that is acceptable for parenteral administration.

Factors affecting the preferred dosage may include, for example, the parasite species infection to be treated and the development stages of the parasites, the type (e.g., species and breed), age, size, sex, diet, activity, and condition of the of the infected animal; the dosage route; pharmacological considerations, such as the activity, efficacy, pharmacokinetic, and toxicology profiles of the particular composition administered; and whether the salt according to this invention being administered as part of a combination of active ingredients. Thus, the preferred amount of the salt according to this invention can vary, and, therefore, can deviate from the typical dosages set forth above. Determining such dosage adjustments is generally within the skill of those in the art.

In a preferred embodiment the salts according to this invention are used to treat a helminth infection caused by one or more helminths selected from the group consisting of a) cestodes: e.g. Anaplocephala spp.; Dipylidium spp.; Diphyllobothrium spp.; Echinococcus spp.; Moniezia spp.; Taenia spp.; b) trematodes e.g. Dicrocoelium spp.; Fasciola spp.; Paramphistomum spp.; Schistosoma spp.; or c) nematodes, e.g.; Ancylostoma spp.; Anecator spp.; Ascaridia spp.; Ascaris spp.; Brugia spp.; Bunostomum spp.; Capillaria spp.; Chabertia spp.; Cooperia spp.; Cyathostomum spp.; Cylicocyclus spp.; Cylicodontophorus spp.; Cylicostephanus spp.; Craterostomum spp.; Dictyocaulus spp.; Dipetalonema spp; Dirofilaria spp.; Dracunculus spp.; Enterobius spp.; Filaroides spp.; Habronema spp.; Haemonchus spp.; Heterakis spp.; Hyostrongylus spp.; Metastrongylus spp.; Meullerius spp. Necator spp.; Nematodirus spp.; Nippostrongylus spp.; Oesophagostomum spp.; Onchocerca spp.; Ostertagia spp.; Oxyuris spp.; Parascaris spp.;Stephanurus spp.; Strongylus spp.; Syngamus spp.; Toxocara spp.; Strongyloides spp.; Teladorsagia spp.; Toxascaris spp.; Trichinella spp.; Trichuris spp.; Trichostrongylus spp.; Triodontophorous spp.; Uncinaria spp., and/or Wuchereria spp.;

It is contemplated that the salts according to this invention may be used to treat animals, including humans and non-human animals, especially non-human mammals. Such non-human mammals include, for example, livestock mammals (e.g., swine, livestock ruminats like bovines, sheep, goats, etc.), laboratory mammals (e.g., mice, rats, jirds, etc.), companion mammals (e.g., dogs, cats, equines, etc.), and wild and zoo mammals (e.g., buffalo, deer, etc.). It is contemplated that the salts according to this invention also are suitable to treat non-mammals, such as poultry (e.g., turkeys, chickens, ducks, etc.) and fish (e.g., salmon, trout, koi, etc.).

In some embodiments, a salt according to this invention is used to treat an infection by a helminth, such as a nematode, cestode or trematode, preferably a nematode (such as Haemonchus contortus), that is resistant to one or more other anthelmintic agents. In some embodiments, the salt according to this invention is active against a helminth, such as a nematode, cestode or trematode, preferably a nematode such as Haemonchus contortus, that is resistant to one or more of the following anthelmintics: an avermectin (e.g., ivermectin, selamectin, doramectin, abamectin, and eprinomectin); a milbemycin (moxidectin and milbemycin oxime); a probenzimidazole (e.g., febantel, netobimin, and thiophanate); a benzimidazole derivative, such as a thiazole benzimidazole derivative (e.g., thiabendazole and cambendazole) or a carbamate benzimidazole derivative (e.g., fenbendazole, albendazole (oxide), mebendazole, oxfendazole, parbendazole, oxibendazole, flubendazole, and triclabendazole); an imidazothiazole (e.g., levamisole and tetramisole); a tetrahydropyrimidine (morantel and pyrantel), an organophosphate (e.g., trichlorphon, haloxon, dichlorvos, and naphthalophos); a salicylanilide (e.g., closantel, oxyclozanide, rafoxanide, and niclosamide); a nitrophenolic compound (e.g., nitroxynil and nitroscanate); benzoenedisulphonamide (e.g., clorsulon); a pyrazinaisoquinoline (e.g., praziquantel and epsiprantel); a heterocyclic compound (e.g., piperazine, diethylcarbamazine, dichlorophen, and phenothiazine); an arsenical (e.g., thiacetarsamide, melorsamine, and arsenamide); cyclooctadepsipeptide (e.g., emodepside); and a paraherquamide

In some such embodiments, for example, the salt according to this invention is active against a helminth (for example, Haemonchus contortus) resistant to an avermectin, such as ivermectin. In other embodiments, the salt according to this invention is alternatively or additionally active against a helminth (for example, Haemonchus contortus) resistant to a benzimidazole derivative, such as fenbendazole. In other embodiments, the salt according to this invention is alternatively or additionally active against a helminth (for example, Haemonchus contortus) resistant to levamisole. And, in other embodiments, the salt according to this invention is alternatively or additionally active against a helminth (for example, Haemonchus contortus) resistant to pyrantel.

The salts according to this invention may be administered in various dosage forms. The term “dosage form” means that the salts according to this invention are formulated into a product suitable for administering to the animal via the envisaged dosage route. Such dosage forms are sometimes referred to herein as formulations or pharmaceutical composition.

The formulation type chosen for a dosage form in any instance will depend upon the particular purpose envisaged and the physical, chemical and biological properties of the salt according to this invention.

Dosage forms useful in the current invention can be liquid, semi-solid or solid dosage forms.

Liquid dosage forms of the salts are generally solutions, suspensions or emulsions. A solution is a mixture of two or more components that form a single phase that is homogeneous down to the molecular level. A suspension consists of insoluble solid particles dispersed in a liquid medium, with the solid particles accounting for about 0.5% to about 30% of the suspension. The liquid may be aqueous, oily, or both. An emulsion is a heterogeneous dispersion of one immiscible liquid in another; it relies on an emulsifying agent for stability. A dry powder (or granule) for reconstitution is reconstituted as a solution or as a suspension immediately prior to injection. The principal advantage of this dosage form is that it overcomes the problem of instability in solution or suspension.

One possible dosage route is the oral dosage route, wherein the salt according to this invention is administered via the mouth. Oral dosage forms suitable for oral administration comprise liquids (e.g. drench or drinking water formulations), semisolids (e.g. pastes, gels), and solids (e.g. tablets, capsules, powders, granules, chewable treats, premixes and medicated blocks).

A drench is a liquid oral formulation that is administered directly into the mouth/throat of an animal, especially a livestock animal, by means of a “drench gun” or syringe or another suitable device. When the composition is administered in the animal recipient's drinking water or as a drench, it may be convenient to use a solution or suspension formulation. This formulation can be, for example, a concentrated suspension that is mixed with water or a dry preparation that is mixed and suspended in the water.

Semi-solid oral formulations (pastes or gels) are generally administered via an applicator directly into the mouth of an animal or mixed with the feed.

Solid oral formulations are either administered directly to an animal (tablet, capsule) or mixed with the feed or via medicated feed blocks.

When the oral formulation is administered via a non-human animal's feed, it may, for example, be fed as a discrete feed or as a chewable treat. Alternatively (or additionally), it may, for example, be intimately dispersed in the animal recipient's regular feed, used as a top dressing, or in the form of solid pellets, paste or liquid that is added to the finished feed. When the oral formulation is administered as a feed additive, it may be convenient to prepare a “premix” in which the oral formulation is dispersed in a liquid or solid carrier. This “premix” is, in turn, dispersed in the animal's feed using, for example, a conventional mixer.

Several modified-release delivery systems have been developed, that take advantage of the unique anatomy of the ruminant forestomach, i.e. for intra-ruminal administration. An intraruminal bolus is a specific formulation for ruminants (cattle, sheep, goats, buffalos, camelids, deer etc). It is a veterinary delayed release delivery system which remains in the rumeno-reticular sac of a ruminant animal over an extended period of time and in which the therapeutically active substance has a predictable and delayed release pattern. Such intraruminal boluses are usually administered using a balling gun or another suitable device.

It is contemplated that the salts according to this invention may alternatively be administered via non-oral dosage routes, such as topically (e.g., via a spot-on, pour-on or transdermal patch), or parenterally (e.g., subcutaneous injection, intravenous injection, intramuscular injection, etc.).

For instance the salts according to this invention may be administered topically using a transdermal formulation (i.e. a formulation that passes through the skin). Alternatively the salts according to this invention may be administered topically via the mucosa.

Topical dosage forms suitable for topical administration comprise liquids (e.g. bath, spray, spot-on), semi-solids (e.g. creams, gels), and solids (e.g. patches, powders, collars). Typical topical formulations for animals are liquid or semi-liquid dosage forms. Typical formulations for transdermal and mucosal administration include, for example, pour-ons, spot-ons, dips, sprays, mousses, shampoos, powders, gels, hydrogels, lotions, solutions, creams, ointments, dusting powders, dressings, foams, films, skin patches, limb bands, collars, ear tags, wafers, sponges, fibers, bandages, and microemulsions. When a liquid formulation is used topically on skin, it can be administered by, for example, pouring on (pour-on or spot-on), spreading, rubbing, atomizing, spraying, dipping, bathing, or washing.

The pour-on or spot-on methods, for example, comprise applying the formulation to a specific location of the skin or coat, such as on the neck or backbone of the animal. This may be achieved by, for example, applying a swab or drop of the pouron or spot-on formulation to a relatively small area of the recipient animal's skin or coat (i.e., generally no greater than about 10% of the animal recipient's skin or coat). In some embodiments, the salt according to this invention is dispersed from the application site to wide areas of the fur due to the spreading nature of the components in the formulation and the animal's movements while, in parallel, being absorbed through the skin and distributed via the animal recipient's fluids and/or tissues.

Parenteral formulations and delivery systems for non-oral routes comprise liquids (e.g. solutions, suspensions, emulsions, and dry powders for reconstitution), semi-solids and solids (e.g. implants). The majority of implants that are used in veterinary medicine are compressed tablets or dispersed matrix systems in which the drug is uniformly dispersed within a nondegradable polymer or alternatively extrusion products.

The methods and pharmaceutical formulations of this invention encompass methods wherein a salt according to this invention is the sole active ingredient administered to the recipient animal. It is contemplated, however, that the methods and pharmaceutical formulations also encompass combination therapies wherein a salt according to the invention is administered in combination with one or more other pharmaceutically acceptable active ingredients. The other active ingredient(s) may be, for example, one or more other salts according to this invention. Alternatively (or additionally), the other active ingredient(s) may be one or more pharmaceutically acceptable compounds that are not salts according to this invention. The other active ingredient(s) may target the same and/or different parasites and conditions.

Contemplated active ingredient(s) that may be administered in combination with the salts include, for example, pharmaceutically acceptable anthelmintics, insecticides and acaricides, insect growth regulators, anti-inflammatories, anti-infectives, hormones, dermatological preparations (e.g., antiseptics and disinfectants), and immunobiologicals (e.g., vaccines and antisera) for disease prevention.

Therefore this invention is also directed to the use as a medicament of combinations comprising
a) one or more salts according to this invention and/or embodiments thereof with
b) one or more pharmaceutically acceptable active compounds which differ in structure from component a). The active compounds b) are preferably anthelmintic compounds, more preferably selected from the group consisting of avermectins (e.g., ivermectin, selamectin, doramectin, abamectin, and eprinomectin); milbemycins (moxidectin and milbemycin oxime); pro-benzimidazoles (e.g., febantel, netobimin, and thiophanate); benzimidazole derivatives, such as a thiazole benzimidazole derivative (e.g., thiabendazole and cambendazole) or a carbamate benzimidazole derivatives (e.g., fenbendazole, albendazole (oxide), mebendazole, oxfendazole, parbendazole, oxibendazole, flubendazole, and triclabendazole); an imidazothiazoles (e.g., levamisole and tetramisole); a tetrahydropyrimidine (morantel and pyrantel), organophosphates (e.g., trichlorphon, haloxon, dichlorvos, and naphthalophos); salicylanilides (e.g., closantel, oxyclozanide, rafoxanide, and niclosamide); nitrophenolic compounds (e.g., nitroxynil and nitroscanate); benzenedisulphonamides (e.g., clorsulon); pyrazineisoquinolines (e.g., praziquantel and epsiprantel); heterocyclic compounds (e.g., piperazine, diethylcarbamazine, dichlorophen, and phenothiazine); arsenicals (e.g., thiacetarsamide, melorsamine, and arsenamide); cyclooctadepsipeptides (e.g., emodepside); paraherquamides (e.g. derquantel); and amino-acetonitrile compounds (e.g. monepantel, AAO 1566); tribendimidine (amidine compound); amidine compounds (e.g., amidantel and tribendimidin), including all pharmaceutically acceptable forms, such as salts, solvates or N-oxides.

Preferred combinations are comprising
a) one salt selected from the group of compounds 1-6 and
b) one compound selected from the group consisting of anthelmintic avermectins (e.g., ivermectin, selamectin, doramectin, abamectin, emamectin and eprinomectin); milbemycins (moxidectin and milbemycin oxime); pro-benzimidazoles (e.g., febantel, netobimin, and thiophanate); benzimidazole derivatives, such as thiazole benzimidazole derivatives (e.g., thiabendazole and cambendazole), carbamate benzimidazole derivatives (e.g., fenbendazole, albendazole (oxide), mebendazole, oxfendazole, parbendazole, oxibendazole, flubendazole, and triclabendazole); imidazothiazoles (e.g., levamisole and tetramisole); tetrahydropyrimidines (morantel and pyrantel), organophosphates (e.g., trichlorphon, haloxon, dichlorvos, and naphthalophos); salicylanilides (e.g., closantel, oxyclozanide, rafoxanide, and niclosamide); nitrophenolic compounds (e.g., nitroxynil and nitroscanate); benzenedisulphonamides (e.g., clorsulon); pyrazineisoquinolines (e.g., praziquantel and epsiprantel); heterocyclic compounds (e.g., piperazine, diethylcarbamazine, dichlorophen, and phenothiazine); arsenicals (e.g., thiacetarsamide, melorsamine, and arsenamide); cyclooctadepsipeptides (e.g., emodepside); paraherquamides (e.g. derquantel); aminoacetonitrile compounds (e.g. monepantel, AAO 1566); tribendimidine (amidine compound); and amidantel (amidine compound); including all pharmaceutically acceptable forms, such as salts.

Preferred combinations are comprising
a) one salt selected from the group of compounds 4 and 5 and
b) one compound selected from the group consisting of anthelmintic avermectins (e.g., ivermectin, selamectin, doramectin, abamectin, emamectin and eprinomectin); milbemycins (moxidectin and milbemycin oxime); pro-benzimidazoles (e.g., febantel, netobimin, and thiophanate); benzimidazole derivatives, such as thiazole benzimidazole derivatives (e.g., thiabendazole and cambendazole), carbamate benzimidazole derivatives (e.g., fenbendazole, albendazole (oxide), mebendazole, oxfendazole, parbendazole, oxibendazole, flubendazole, and triclabendazole); imidazothiazoles (e.g., levamisole and tetramisole); tetrahydropyrimidines (morantel and pyrantel), organophosphates (e.g., trichlorphon, haloxon, dichlorvos, and naphthalophos); salicylanilides (e.g., closantel, oxyclozanide, rafoxanide, and niclosamide); nitrophenolic compounds (e.g., nitroxynil and nitroscanate); benzenedisulphonamides (e.g., clorsulon); pyrazineisoquinolines (e.g., praziquantel and epsiprantel); heterocyclic compounds (e.g., piperazine, diethylcarbamazine, dichlorophen, and phenothiazine); arsenicals (e.g., thiacetarsamide, melorsamine, and arsenamide); cyclooctadepsipeptides (e.g., emodepside); paraherquamides (e.g. derquantel); aminoacetonitrile compounds (e.g. monepantel, AAO 1566); tribendimidine (amidine compound); and amidantel (amidine compound); including all pharmaceutically acceptable forms, such as salts.

Preferred combinations comprise at least one salt selected from the group of compounds 1-6 and
abamectin, ivermectin, emamectin, eprinomectin, doramectin, moxidectin,
milbemycin oxime; or.
closantel, oxyclozanide, rafoxanide, niclosamide; or
nitroxynil, nitroscanate, clorsulon; or
praziquantel, epsiprantel; or
emodepside, derquantel, monepantel.

The salts of the current invention can be combined with pharmaceutically acceptable insecticides or acaricides. Such pharmaceutically acceptable insecticides and acaricides include, for example, acetamiprid, acetoprole, amitraz, amidoflumet, avermectin, azadirachtin, bifenthrin, bifenazate, buprofezin, bistrifluron, chlorfenapyr, chlorfluazuron, chlorantraniliprole, chlorpyrifos, chromafenozide, clothianidin, cyantraniliprole, cyflumetofen, 13-cyfluthrin, cyhalothrin, A. cyhalothrin, cymiazole cypermethrin, cyromazine, deltamethrin, demiditraz, diafenthiuron, diazinon, diflubenzuron, dimefluthrin, dinotefuran, emamectin, esfenvalerate, ethiprole, fenoxycarb, fenpropathrin, fenvalerate, fipronil, flonicamid, flubendiamide, flucythrinate, tau-fluvalinate, flufenoxuron, halofenozide, hexaflumuron, imidacloprid, indoxacarb, lufenuron, metaflumizone, methoprene, metofluthrin, methoxyfenozide, nitenpyram, novaluron, noviflumuron, permethrin, phosmet, profluthrin, protrifenbute, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen, spirotetramat, sulfoxaflor, tebufenozide, tebufenpyrad, teflubenzuron, tefluthrin, tetrachlorvinphos, tetramethylfluthrin, thiacloprid, thiamethoxam, tolfenpyrad, tralomethrin, and triflumuron. General references discussing antiparasitic agents, such as insecticides and acaricides, include, for example, The Pesticide Manual, 13th Edition, C. D.S. Tomlin, Ed., British Crop Protection Council, Farnham, Surrey, U.K. (2003).

In some contemplated embodiments, the salts are administered with pyridylmethylamine derivatives, such as, for example, pyridylmethylamine derivatives discussed in European Patent Appl. EP0539588 or Int'l Patent Appl. Publ. W02007/115643.

In some contemplated embodiments, the salts is administered with nodulisporic acids and derivatives thereof, such as, for example, compounds discussed in U.S. Pat. Nos. 5,399,582; 5,945,317; 5,962,499; 5,834,260; 6,221,894; or 5,595,991; or Int'l Patent Appl. Publ. 1996/29073.

Pharmaceutically acceptable insect growth regulators include, for example, methoprene, pyriproxyfen, tetrahydroazadirachtin, chlorfluazuron, cyromazine, diflubenzuron, fluazuron, flucycloxuron, flufenoxuron, hexaflumuron, lufenuron, ifenuron, tebufenozide, and triflumuron. These compounds tend to provide both initial and sustained treatment of parasite infections at all stages of insect development, including eggs, on the animal subject, as well as within the environment of the animal subject.

Other antiparasitic compounds contemplated to be useful in combination therapies with the salts of the invention include, for example, imidazo[1,2-b] pyridazine compounds discussed in US Patent Appl. Publ. No. 2005-0182059; 1-(4-Mono and dihalomethylsulphonylphenyl)-2-acylamino-3-fluoropropanol compounds discussed U.S. Pat. No. 7,361,689; trifluoromethanesulfonanilide oxime ether compounds discussed in U.S. Pat. No. 7,312,248; n-[(phenyloxy)phenyl]-1, 1, 1-trifluoromethanesulfonamide and n[(phenylsulfanyl)phenyl]-1, 1, 1-trifluoromethanesulfonamide compounds discussed in US Patent Appl. Publ. 2006-0281695; and 2-phenyl-3-(1H-pyrrol-2-yl)acrylonitrile compounds discussed in US Appl. Publ. 2006/0128779; isoxazoline compounds discussed in WO Patent Appl, Publ. 2005-085216, WO 2007-026965, WO 2007-070606, WO 2007-075459, WO 2007-079162, WO 2007-105814, WO 2007-125984, WO 2008-019760, WO 2008-122375, WO 2008-150393, WO 2009-002809, WO 2009-003075, WO 2009-022746, WO 2009-035004, WO 2009-045999, WO 2009-051956, WO 2009-035004.

In the contemplated combination therapies, the salts according to this invention may be administered before, simultaneously, and/or after the other active ingredient(s). In addition, the salts according to this invention may be administered in the same composition as the other active ingredient(s) and/or in separate compositions from the other active ingredient(s). Further, the salts according to this invention and other active ingredient(s) may be administered via the same and/or different dosage route.

When the salts according to this invention are administered in a combination therapy, the weight ratio of the active ingredients may vary widely. Factors influencing this ratio include, for example, the particular salts; the identity of the other active ingredient(s) be administered in the combination therapy; the dosage route of the salts and other active ingredient(s); the target condition and pathogen; the type (e.g., species and breed), age, size, sex, diet, activity, and condition of the animal; and pharmacological considerations, such as the activity, efficacy, pharmacokinetic, and toxicology profiles of the salts and other active ingredient(s). In some contemplated embodiments, for example, the weight ratio of the salts to the other active ingredient(s) is, for example, is from about 1:3000 to about 3000:1. In some such instances, the weight ratio is from about 1:300 to about 300:1. In other such instances, the weight ratio is from about 1:30 and about 30:1.

In addition to other active ingredients, it is contemplated that the salts may be administered with one or more other compounds that beneficially affects (e.g. enhances or prolongs) the activity (or other characteristic, such as safety) of the salts. For example, it is contemplated that the salts may be administered with one or more synergists, such as, for example, piperonyl butoxide (PBO) and triphenyl phosphate (TPP). Other synergists include, for example, N-(2-ethylhexyl)-8,9,10-trinorborn-5-ene-2,3-dicarboxamide, (also known as “ENT 8184” or “MGK 264”) and Verbutin (also known as “MB-599”).

The salts used according to this invention show an excellent activity in treating parasite infections and in addition are acceptable for the animals treated.

The invention will now be further described by the following, non-limiting, examples.

### EXAMPLES

Compounds of formula (I) may be synthesized as described in WO2012/041873

| Compound # | |
|---|---|
| 1 | 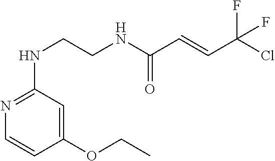 [SMILES: CCOc1ccnc(NCCNC(=O)/C=C/C(F)(F)Cl)c1] |
| 2 | 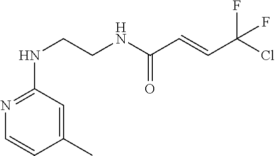 [SMILES: Cc1ccnc(NCCNC(=O)/C=C/C(F)(F)Cl)c1] |
| 3 | 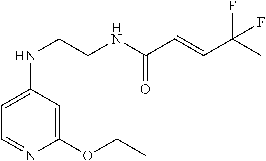 [SMILES: CCOc1cc(NCCNC(=O)/C=C/C(C)(F)F)ccn1] |
| 4 | 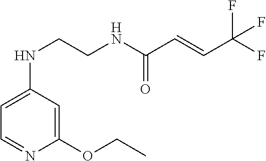 [SMILES: CCOc1cc(NCCNC(=O)/C=C/C(F)(F)F)ccn1] |
| 5 | 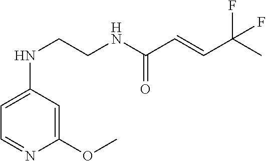 [SMILES: COc1cc(NCCNC(=O)/C=C/C(C)(F)F)ccn1] |
| 6 | 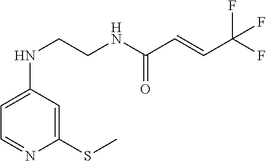 [SMILES: CSc1cc(NCCNC(=O)/C=C/C(F)(F)F)ccn1] |

### 1. Example 1

### Salts of Compound 4

### 1A. Synthesises of the tartrate salt of (E)-N-[2-[(2-ethoxy-4-pyridyl)amino]ethyl]-4,4,4-trifluoro-but-2-enamide (Compound 4)

(E)-N-[2-[(2-ethoxy-4-pyridyl)amino]ethyl]-4,4,4-trifluoro-but-2-enamide (5.0 g, 0.0165 mol) was dissolved in 2-propanol with stirring. A solution of L-tartaric acid (2.5 g, 0.0165 mol) in a 1:1 mixture of water and 2-propanol was added and the resulting solution was stirred at room temperature for 30 min. Seed crystals of the tartrate salt of (E)-N-[2-[(2-ethoxy-4-pyridyl)amino]ethyl]-4,4,4-trifluoro-but-2-enamide were added and the crystallization started after 5 min. The resulting slurry was filtered, the filter cake was washed with 2-propanol (100 ml) and the resulting solid was dried under reduced pressure.

¹H-NMR (400 MHz, DMSO-d₆): δ (ppm) 8.66 (1H, t, J=5.5 Hz), 7.63 (1H, d, J=5.9 Hz), 6.7 (2H, m), 6.60 (1H, t, J=5.7 Hz), 6.22 (1H, dd, J=5.9 Hz, 2 Hz), 5.85 (1H, J=1.9 Hz), 4.30 (2H, s), 4.19 (2H, q, J=7.0 Hz), 3.78 (1H, qn, J=6.1 Hz), 3.31 (2H, q, J=6.2 Hz), 3.18 (2H, J=6.2 Hz), 1.25 (3H, t, J=7.0 Hz), 1.04 (6H, d, J=6.1 Hz)

A synthesized tartrate salt of compound 4 can be dissolved with 145 mg/ml. The free base of compound 4 has a solubility of 0.8 mg/ml

### 1B. Solubility of Salts of Compound 4

A defined amount of an acid (1.0 g) was weighed into a flask and dissolved in water (10 ml). Compound 4 was added in portions of 1.0 or 0.5 grams with stirring, after each addition it was checked if the addition resulted in a clear solution or if precipitation occurred. The pH value was measured after each addition. The following table shows the amount of compound 4 that can be added to the solution of each acid in 10 ml water resulting in a clear solution and the corresponding pH value of this solution.

| acid | Dissolved g of 4 | pH |
|---|---|---|
| Tartaric acid | 3.5 | 4.2 |
| Maleic acid | <0.1 | n.m.# |
| Citric acid | 2.5 | 4.4 |
| Acetic acid* | 1 | 4.6 |

*0.5 g of Acetic Acid used
#Not Measured

In addition, 2 g of compound 4 and 1 g of tartaric acid was dissolved in 2.5 ml of water and gives a clear solution. Thus 8 g of compound 4 in 10 ml of water can be dissolved.

1C. Stability Tests of Solutions

Equimolar amounts of compound 4 and acids were diluted with water: Water was added until all residues were dissolved. 200, 100 and 50 mg of compound 4 was used, each amount was combined with each acid of the following list. The solutions were equilibrated at room temperature and then held for 7 days at room temperature and thereafter 7 days at 8° C.

The following acids were used:

| | | |
|---|---|---|
| hydrochloric | p-toluenesolfonic | methanesulfonic |
| acid | acid | acid |
| benzenesulfonic | Phosphoric acid | ethanesulfonic |
| acid | | acid |
| sachcharin | salicylic acid | lactic acid |
| benzoic acid | acetic acid | ethanedisulfonic |
| | | acid |
| oxalic | sulfuric acid | ethane-1,2- |
| | | disulfonic acid |
| maleic acid | malonic acid | tartaric acid |
| fumaric acid | succinic acid | citric acid |
| 1,5-naphthalene | disulfonic | isethionic acid |
| acid | | |

The following acids show solubility of >200 mg/ml

| Acid | pH |
|---|---|
| Hydrochloric | 1.15 |
| Sulfuric | 0.06 |
| Ethanesulfonic | 1.23 |
| Methanesulfonic | 1.28 |
| Oxalic | 2.14 |
| Tartaric | 3.35 |
| Fumaric | 3.74 |
| Citric | 3.41 |
| isethionic acid | |

The following acids show solubility of >100 mg/ml

| Acid | pH |
|---|---|
| D-glucoronic | 4.15 |
| L-lactic | 4.28 |

Phosphoric acid

The following acids show solubility of <50 mg/ml

Acid

ethanedisulfonic acid,

maleic acid,

malonic acid,

benzoic acid,

succinic acid,

acetic acid,

p-toluenesulfonic acid,

benzenesulfonic acid,

saccharin

### Example 2

### 2A. Stability Tests of Solutions of Compound 5

A visual solubility test (100 μL) at a series of target concentrations of 50, 100 or 200 mg/mL of compound 5 in aqueous acid solution was performed.

In a 96 well plate (400-600 μL), compound 5 was dispensed (5, 10, or 20 mg per well), acid was added (100 μl of a 350 mM solution in water to each well) and the mixtures were equilibrated at 25° C. with stirring. Solubility was checked by visual inspection after 30 min and after one day.

The following amounts of compounds could be dissolved to give a clear solution:

| | Range at | Range at | pH for | pH for | pH for |
|---|---|---|---|---|---|
| | 30 mins. | 1 Day | 50 | 100 | 200 |
| Solution | mg/mL | mg/mL | mg/mL | mg/mL | mg/mL |
| Tartaric Acid | 100 < x < 200 | 100 < x < 200 | 2.92 | 3.71 | 4.70 |
| Fumaric Acid | 104 < x < 202 | 104 < x < 200 | 3.59 | 3.89 | 4.86 |
| Citric Acid | 98 < x < 203 | 99 < x < 203 | 3.36 | 4.06 | 4.84 |
| Malic Acid | 101 < x < 203 | 101 < x < 203 | 3.68 | 4.45 | 4.78 |
| Oxalic Acid | 105 < x < 209 | 105 < x <2 09 | 1.81 | 3.27 | 4.81 |
| Lactic Acid | 50 < x < 102 | 50 < x < 102 | 4.50 | 4.88 | 4.94 |
| Glucuronic | 51 < x < 107 | 51 < x < 107 | 3.60 | 4.86 | 4.80 |
| Acid | | | | | |
| Oxoglutaric | 104 < x < 199 | 104 < x < 199 | 2.66 | 4.17 | 4.89 |
| Acid | | | | | |
| Nicotinic Acid | <74 | <74 | 5,25 | 5.15 | 5.16 |
| Ethanesulfonic | 104 < x < 197 | 104 < x < 197 | 0.96 | 1.17 | 4.99 |
| Acid | | | | | |
| Methanesulfonic | <52 | <52 | 7.60 | 7.61 | 7.31 |
| Acid | | | | | |
| lsethionic Acid | <52 | <52 | 7.83 | 7.85 | 7.93 |
| Benzoic Acid | <49 | 49 < x < 109 | 4.94 | 5.23 | 5.20 |
| Succinic Acid | 48 < x <100 | 48 < x < 100 | 4.57 | 5.01 | 4.99 |
| Acetic Acid | <58 | <58 | 5.09 | 5.02 | 5.06 |
| Glutamic Acid | <53 | <53 | 5.11 | 5.05 | 5.14 |

As can be seen for compound 5, tartaric acid, fumaric acid, citric acid, malic acid, oxalic acid, oxoglutaric acid and ethanesulfonic acid provided good solubility. Nicotinic acid, methanesulfonic acid, isethionic acid benzoic acid, acetic acid and glutamic acid gave poor solubility.

### 2B. Quantitative Determination of Solubility of Compound 5

For the acids that showed good solubility in Example 2A, an extended screen was performed to see how the solutions performed over 3 days:

Compound 5 (120 mg) was weighed into shell vials and combined with acid (600 μl of a 350 mM solution of the respective acid in water). The vials were rotated at 25° C. and assayed at 1 and 3 days: at each time point 200 μl were sampled, filtered through a Millipore solubility plate (0.4 μM polycarbonate filter), The amount of compound 5 in the filtrate was quantified by UPLC.

Solubility of Compound 5:

| | T = 1 Day | | T = 3 Days | |
|---|---|---|---|---|
| | | Average | | Average |
| Solution | mg/mL | (mg/mL) | mg/mL | (mg/mL) |
| Tartaric | 117.1 | 137.2 | 161.7 | 163.0 |
| Fumaric | 151.6 | 148.8 | 139.7 | 152.6 |
| Citric | 159.5 | 149.3 | 152.9 | 150.8 |
| Malic | 128.8 | 121.8 | 116.8 | 116.5 |
| Oxalic | 149.2 | 142.0 | 155.2 | 155.2 |
| Oxoglutaric | 127.5 | 129.3 | 134.6 | 133.5 |
| Ethanesulfonic | 154.8 | 144.6 | 167.4 | 164.8 |

These 7 acids were capable of dissolving compound 5 between 100 and 200 mg/ml

### 2C. Formulation with 10% ‘Compound 5

A formulation with 10% of compound 5 (˜100 mg/ml) was prepared with pH of 4.4 and stability (remains clear solution) for 3 months with equimolar amounts of the following acids:
Tartaric acid
Malic acid
Citric acid

### Example 3 Formulation Studies

The following formulations were prepared and administered to animals. Blood samples were collected at pre-defined time points and the amount of compound present in plasma at the time of sampling was determined by HPLC-MS/MS. Pharmacokinetic parameters were then calculated: Cmax, Tmax, Tlast, AUC.

| | | |
|---|---|---|
| FormA₅₀ | Compound | 50 mg |
| | N-methyl pyrrolidone (NMP) | 0.65 mL |
| | Propylene glycol | 0.15 mL |
| | Water for injections | Up to 1 mL |
| FormS₅₀ | Compound | 50 mg |
| | L-tartaric acid, anhydrous | 24.74 mg |
| | Sodium carboxymethyl | 3 mg |
| | cellulose (medium viscosity) | |
| | 2-Pyrrolidone | 0.4 mL |
| | 1 M Sodium hydroxide solution | 0.12 mL |
| | Water for injections | Up to 1.0 mL |
| FormO₅₀ | Compound | 50 mg |
| | L-tartaric acid, anhydrous | 24.74 mg |
| | 1 M Sodium hydroxide solution | Up to pH 4.4 |
| | Water for injections | Up to 1 mL |
| FormO_{33.3} | Compound | 33.3 mg |
| | L-tartaric acid, anhydrous | 16.50 mg |
| | 1 M Sodium hydroxide solution | Up to pH 4.4 |
| | Water for injections | Up to 1 mL |
| FormO_{16.7} | Compound | 16.7 mg |
| | L-tartaric acid, anhydrous | 8.25 mg |
| | 1 M Sodium hydroxide solution | Up to pH 4.4 |
| | Water for injections | Up to 1 mL |
| FormO₁₀₀ | Compound | 100 mg |
| | L-tartaric acid, anhydrous | 52.6 mg |
| | 5 M Sodium hydroxide solution | Up to pH 4.4 |
| | Water for injections | Up to 1 mL |
| | 2.5% IV Solution Compound | 25.0 mg |
| | Dimethyl acetamide | Up to 1 mL |
| | 1% IV Solution Compound | 10 mg |
| | Dimethyl acetamide | Up to 1 mL |
| FormB_{62.5} | Compound | 62.5 mg |
| | Dimethyl acetamide | 0.65 mL |
| | Propylene glycol | 0.15 mL |
| | Water for injections | Up to 1 mL |
| FormC_{62.5} | Compound | 62.5 mg |
| | Propylene glycol | 0.4 mL |
| | Ethanol | 0.1 mL |
| | Diethylene glycol monoethyl ether | Up to 1 mL |
| | (Transcutol P) | |
| FormD_{62.5} | Compound | 62.5 mg |
| | Polyethylene glycol 300 (PEG 300) | 0.4 mL |
| | Ethanol | 0.1 mL |
| | Diethylene glycol monoethyl ether | Up to 1mL |
| | (Transcutol P) | |
| FormE_{62.5} | Compound | 62.5 mg |
| | Polyethylene glycol 300 | 0.5 mL |
| | Ethyl alcohol 96% | 0.3 mL |
| | Water for injections (WFI) | Up to 1mL |
| FormF_{62.5} | Compound | 62.5 mg |
| | Polyethylene glycol | 0.5 mL |
| | 1-Octanol | 0.25 mL |
| | 2-Pyrrolidone | Up to 1.0 mL |
| FormG_{62.5} | Compound | 62.5 mg |
| | Benzyl alcohol | 0.25 mL |
| | Ethyl alcohol 96% | 0.1 mL |
| | Polyethylene glycol 300 | Up to 1.0 mL |
| FormH_{62.5} | Compound | 62.5 mg |
| | Dimethyl acetamide (DMA) | 0.3 mL |
| | Dimethyl sulfoxide (DMSO) | 0.5 mL |
| | 2-Pyrrolidone | Up to 1.0 mL |
| FormJ_{62.5} | Compound | 62.5 mg |
| | Dimethyl sulfoxide (DMSO) | 0.1 mL |
| | 2-Pyrrolidone | 0.5 mL |
| | Ethyl lactate | Up to 1.0 mL |
| FormK_{62.5} | Compound | 62.5 mg |
| | 2-Pyrrolidone | 0.4 mL |
| | Diethylene glycol monoethyl ether | 0.4 mL |
| | (Transcutol P) | |
| | Ethyl lactate | ad 1.0 mL |
| FormL₅₀ | Compound | 50 mg |
| | Dimethyl acetamide (DMA) | 0.65 mL |
| | Propylene glycol (PG) | 0.15 mL |
| | Water for injections (WFI) | Up to 1.0 mL |
| FormM_{62.5} | Compound | 62.5 mg |
| | Anhydrous L-tartaric acid | 30.93 mg |
| | Sodium hydroxide | up to pH 4.6 |
| | Water for injections (WFI) | Up to 1.0 mL |
| FormN_{62.5} | Compound | 62.5 mg |
| | Sodium carboxymethyl cellulose | 1 mg |
| | Sodium chloride | 6 mg |
| | Polysorbate 80 | 0.5 mg |
| | Water for injections (WFI) | Up to 1.0 mL |
| FormP_{62.5} | Compound | 62.5 mg |
| | L-tartaric acid, anhydrous | 30.93 mg |
| | 2-pyrrolidone | 0.3 mL |
| | 1 M Sodium hydroxide solution | 0.14 mL |
| | Water for injections | up to 1.0 mL |
| FormQ_{62.5} | Compound | 62.5 mg |
| | L-tartaric acid, anhydrous | 30.93 mg |
| | Sodium carboxymethyl | 1 mg |
| | cellulose (medium viscosity) | |
| | 1 M Sodium hydroxide solution | up to pH 4.3 |
| | Water for injections | up to 1 mL |
| FormR₅₀ | Compound | 50 mg |
| | L-tartaric acid, anhydrous | 24.74 mg |
| | 2-pyrrolidone | 0.4 mL |
| | 1 M Sodium hydroxide solution | 0.12 mL |
| | Water for injections | up to 1 mL |
| FormS₅₀ | Compound | 50 mg |
| | L-tartaric acid, anhydrous | 24.74 mg |
| | Sodium carboxymethyl | 3 mg |
| | cellulose (medium viscosity) | |
| | 2-pyrrolidone | 0.4 mL |
| | 1 M Sodium hydroxide solution | 0.12 mL |
| | Water for injections | up to 1 mL |
| FormT₅₀ | Compound | 50 mg |
| | DL-Lactic acid | 18.56 mg |
| | 1 M hydrochloric acid | up to pH 4.4 |
| | Water for injections | up to 1 mL |
| FormU₁₀₀ | Compound | 100 mg |
| | L-tartaric acid | 52.61 mg |
| | Propylene glycol | 400 mg |
| | Benzylalcohol | 20 mg |
| | Water for injection | Up to 1 mL |
| FormV₁₀₀ | Compound | 100 mg |
| | L-tartaric acid | 52.61 mg |
| | Propylene glycol | 500 mg |
| | Benzylalcohol | 20 mg |
| | Water for injection | Up to 1 mL |
| FormW₁₀₀ | Compound | 100 mg |
| | L-tartaric acid | 52.61 mg |
| | Propylene glycol | 300 mg |
| | Benzylalcohol | 20 mg |
| | Water for injection | Up to 1 mL |

PK Studies:

| | | | | Dose | | | | |
|---|---|---|---|---|---|---|---|---|
| | | | | (mg/ | Cmax | Tmax | Tlast | AUC |
| Compound | Formulation | Route | Animal | kg) | (ng/mL)* | (h)* | (h)* | (h*ng/mL)* |
| 4 | Form A₅₀ | SC | Cattle | 1.25 | 127 | 0.44 | 6.5 | 204 |
| | Form O_{62.5} | SC | Cattle | 1.25 | 428 | 0.31 | 3 | 372 |
| | Form B_{62.5} | SC | Cattle | 1.25 | 37.4 | 0.5 | 5.33 | 98.2 |
| | FormC_{62.5} | SC | Cattle | 1.25 | 46.4 | 0.66 | 10.7 | 130 |
| | FormD_{62.5} | SC | Cattle | 1.25 | 25.4 | 0.92 | 4 | 63.2 |
| | FormE_{62.5} | SC | Cattle | 1.25 | 45.7 | 0.42 | 5 | 76.4 |
| | FormF_{62.5} | SC | Cattle | 1.25 | 15.2 | 0.63 | 8 | 78.5 |
| | FormG_{62.5} | SC | Cattle | 1.25 | 12.5 | 2.33 | 5.33 | 44.8 |
| | FormH_{62.5} | SC | Cattle | 1.25 | 42.1 | 0.67 | 4 | 88.2 |
| | FormJ_{62.5} | SC | Cattle | 1.25 | 37.1 | 1.83 | 4 | 99.6 |
| | FormK_{62.5} | SC | Cattle | 1.25 | 29.9 | 0.83 | 4 | 86 |
| | FormL₅₀ | SC | Cattle | 1.25 | 28.1 | 0.67 | 4 | 59.8 |
| | FormM_{62.5} | SC | Cattle | 1.25 | 349.8 | 0.25 | 3.33 | 335.6 |
| | FormO_{62.5} | SC | Cattle | 1.25 | 300 | 0.5 | 4 | 358 |
| 4 | FormP_{62.5} | SC | Cattle | 1.25 | 94.2 | 0.83 | 6.67 | 322 |
| | FormQ_{62.5} | SC | Cattle | 1.25 | 297 | 0.58 | 4 | 356 |
| | FormO₆₀ | SC | Cattle | 1 | 374 | 0.25 | 2.67 | 302 |
| 4 | FormR₅₀ | SC | Cattle | 1 | 59.4 | 1 | 6.67 | 248 |
| | FormS₅₀ | SC | Cattle | 1 | 69.3 | 1.33 | 8.67 | 339 |
| | FormT₅₀ | SC | Cattle | 1 | 281.4 | 0.42 | 3.67 | 293 |
| 5 | FormO₅₀ | SC | Cattle | 1 | 521 | 0.6 | 6 | 973 |
| | FormO₅₀ | SC | Cattle | 0.75 | 397 | 0.5 | 5.6 | 616 |
| | FormO₅₀ | SC | Cattle | 0.5 | 305 | 0.3 | 4.4 | 371 |
| 5 | FormO₅₀ | SC | Cattle | 1 | 705 | 0.3 | 7 | 1149 |
| | FormO₅₀ | SC | Cattle | 5 | 3309 | 0.4 | 8 | 7887 |
| | FormO₅₀ | SC | Cattle | 10 | 5929 | 0.6 | 12 | 18832 |
| 5 | FormO₅₀ | SC | Cattle | 1 | 815 | 0.5 | 6.8 | 969 |
| | FormO₅₀ | SC | Cattle | 5 | 5975 | 0.4 | 8 | 8817 |
| 5 | FormO₁₀₀ | SC | Cattle | 1.5 | 1010 | 0.31 | 10.0 | 1678 |
| | FormO₁₀₀ | SC | Cattle | 1.5 | 862 | 0.38 | 8.5 | 1422 |
| | FormU₁₀₀ | SC | Cattle | 1.5 | 593 | 0.63 | 10.0 | 1684 |
| | FormV₁₀₀ | SC | Cattle | 1.5 | 540 | 1.75 | 10.0 | 1523 |
| | FormW₁₀₀ | SC | Cattle | 1.5 | 743 | 0.88 | 9.5 | 1523 |

It can be seen that all formulations containing the tartrate or lactate salt show significantly higher Cmax and AUC.

### Experiment 4: In Vivo Study Efficacy of a Formulation of the Tartrate Salt

Calves of 2-6 months of age were challenged with Ostertagia ostertagi (approx. 20,000 larvae L3/calf) on Day 0 and Cooperia oncophora (approx. 20,000 larvae L3/calf) on Day 1 by oral drench. At day 28, animals were treated by subcutaneous injection at a dose of 0.5 mg/kg (resp. 0.75, 1, 1.5 or 2.5 mg/kg) of a solution of compound 5.

On days 35-37 animals were necropsied for extraction and counting of nematodes present. Efficacy was calculated as the percent reduction of nematodes compared to the control group. For calculation the geometric mean of the nematode numbers in treatment and control group was determined (7 animals in treatment group, 8 animals in control group).

The solution A used for injection contained in 1 ml: compound 5 (100 mg), L-tartaric acid 52.6 mg, propylene glycol 400 mg, benzyl alcohol 20 mg, water for injection up to 1 ml. A control group was treated by injection of vehicle only.

The solution B used for injection contained in 1 ml: (50 mg Compound 5; 24.74 mg L-tartaric acid, water for injection up to 1 ml).

Solution A: Formulation with Propylene Glycol

Treatment with tartrate salt of compound 5 at 0.5 mg/kg bodyweight resulted in a reduction of O. ostertagi of 98.7% and of C. oncophora of 97.0%.

Treatment with tartrate salt of compound 5 at 1.5 mg/kg bodyweight resulted in a reduction of O. ostertagi of 100% and of C. oncophora of 100%.

Treatment with tartrate salt of compound 5 at 2.5 mg/kg bodyweight resulted in a reduction of O. ostertagi of 99.8% and of C. oncophora of 100%.

Solution B: Formulation without Propylene Glycol

Treatment with tartrate salt of compound 5 at 0.5 mg/kg bodyweight resulted in a reduction of O. ostertagi of 78.6% and of C. oncophora of 80.4.0%.

Treatment with tartrate salt of compound 5 at 0.75 mg/kg bodyweight resulted in a reduction of O. ostertagi of 99.8% and of C. oncophora of 94.0%.

Treatment with tartrate salt of compound 5 at 1 mg/kg bodyweight resulted in a reduction of O. ostertagi of 99.9% and of C. oncophora of 96.8%.

At lower concentrations it is clear that formulations with propylene glycol have a higher efficacy in reducing helminths in cattle.

1-16. (canceled)

17. A salt of a compound of formula (I) 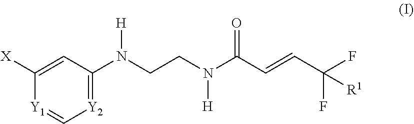
R¹ is halogen or methyl
X is methyl, OR², or SR²
Y₁, Y₂ is CR³ or N, and one of Y₁, Y₂ is N and one of Y₁, Y₂ is CR³
R² is methyl or ethyl
R³ is hydrogen or methyl,
wherein the salt is chosen from the group consisting of malate salt, citrate salt, tartrate salt, oxalate salt, fumarate salt, lactate salt, glucoronate salt, oxoglucoronate salt, ethanesulfonate salt, and succinate salt.

18. The salt according to claim 17, wherein the halogen is fluorine or chlorine.

19. The salt according to claim 18, wherein the halogen is fluorine.

20. The salt according to claim 17, wherein the SR² is S-methyl.

21. The salt according to claim 17, wherein OR² is O-methyl or O-ethyl.

22. The salt according to claim 17, wherein R³ is hydrogen.

23. The salt according to claim 17, wherein Y₁ is N and Y₂ is CH.

24. The salt according to claim 17, wherein the compound of formula (I) is a compound selected from the group consisting of

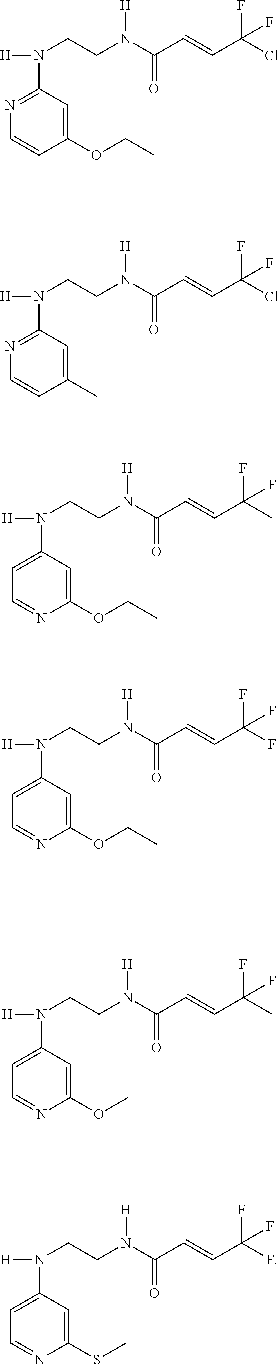 [SMILES: CCOc1ccnc(NCCNC(=O)/C=C/C(F)(F)Cl)c1 ; Cc1ccnc(NCCNC(=O)/C=C/C(F)(F)Cl)c1 ; CCOc1cc(NCCNC(=O)/C=C/C(C)(F)F)ccn1 ; CCOc1cc(NCCNC(=O)/C=C/C(F)(F)F)ccn1 ; COc1cc(NCCNC(=O)/C=C/C(C)(F)F)ccn1 ; CSc1cc(NCCNC(=O)/C=C/C(F)(F)F)ccn1]

25. The salt according to claim 17, wherein the salt is chosen from the group consisting of malate salt, citrate salt, tartrate salt, oxalate salt, fumarate salt, oxoglucoronate salt, and ethanesulfonate salt.

26. The salt according to claim 25, wherein the salt is chosen from the group consisting of malate salt, citrate salt, tartrate salt.

27. A pharmaceutical formulation comprising the salt of claim 17, wherein the base compound is present in an amount of 2-30% (w/v), and wherein the formulation has a pH value of at least 3.7.

28. The pharmaceutical formulation according to claim 27, wherein the formulation is an aqueous formulation.

29. The pharmaceutical formulation according to claim 27, further comprising a preservative.

30. The pharmaceutical formulation according to claim 27, wherein the preservative is benzylalcohol.

31. The pharmaceutical formulation according to claim 27, further comprising an antifreeze compound.

32. The pharmaceutical formulation according to claim 31, wherein the antifreeze compound is selected from the group consisting of propylene glycol, ethylene glycol, and glycerol.

33. The pharmaceutical formulation according to claim 32, wherein the antifreeze compound is propylene glycol.

34. A method for treating a parasitic disease in an animal wherein a salt according to claim 17 is administered to the animal.

35. A method for treating a parasitic disease in an animal wherein the pharmaceutical formulation according to claim 26 is administered to the animal.

36. The salt according to claim 24, wherein the compound is

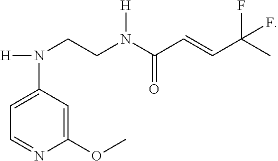 [SMILES: COc1cc(NCCNC(=O)/C=C/C(C)(F)F)ccn1]

37. The salt of claim 36, wherein the salt is the tartrate salt.
